(19) United States
(12) Patent Application Publication
(10) Pub. No.: US 2014/0296780 A1
(43) Pub. Date: Oct. 2, 2014

(54) POLYMER COMPOSITE CARBON NANOTUBE MICRONEEDLE DEVICES AND THEIR FABRICATION

(71) Applicants: Bradley Lyon, Arcadia, CA (US); Adrianus I. Aria, Cambridge (GB); Masoud Beizai, Laguna Hills, CA (US); Morteza Gharib, Altadena, CA (US)

(72) Inventors: Bradley Lyon, Arcadia, CA (US); Adrianus I. Aria, Cambridge (GB); Masoud Beizai, Laguna Hills, CA (US); Morteza Gharib, Altadena, CA (US)

(73) Assignee: CALIFORNIA INSTITUTE OF TECHNOLOGY, Pasadena, CA (US)

(21) Appl. No.: 14/231,267

(22) Filed: Mar. 31, 2014

Related U.S. Application Data
(60) Provisional application No. 61/807,224, filed on Apr. 1, 2013.

(51) Int. Cl. A61M 37/00

(52) U.S. Cl. CPC A61M 37/0015; A61M 2037/003; A61M 2037/0053
USPC 604/103.01; 604/173; 264/279.1

(57) ABSTRACT

Composite CNT microneedle fabrication and finally-constructed delivery device embodiments are described. The microneedles can provide a self-administered, painless alternative to standard hypodermic injection.

### RELATED APPLICATIONS

This application claims the benefit of and priority to U.S. Provisional Patent Application Ser. No. 61/807,224, filed Apr. 1, 2013, which is incorporated by reference herein in its entirety for all purposes.

### FIELD

This application relates to micro-scale and/or nano-scale needles (collectively referred to herein as microneedles), especially carbon nanotube (CNT) microneedles, microneedle arrays, and microneedle array-type devices.

### BACKGROUND

In the last few decades, the development of new drugs with greater potency has been a focus in the pharmaceutical field. However, the administration of such drugs has been limited due to poor absorption and enzymatic degradation in the gastrointestinal tract as well as painful delivery using intravascular injection. One feasible solution to solve these problems is by administering those drugs across the skin using a patch, although the patch's therapeutic rates can be limited due to skin permeability. In recent years, substantial effort has been spent to overcome this difficulty by incorporating skin permeation enhancers, electric fields, ultrasound, and microneedles in the drug delivery systems. Among these approaches, microneedles appear most promising since they create holes to bypass the stratum corneum of a patient's skin with little or no pain.

Microneedles, especially when arranged in arrays, find use in obtaining biological fluid samples and for delivering drugs, agents, formulations, or biological molecules across biological tissue barriers. The microneedles used for delivering one or more compounds may be categorized as luminal or dissolvable. Dissolvable microneedles include a polymer tip that dissolves when in contact with body fluid to deliver a drug, vaccine inoculation, or other therapeutic agent. As the designation implies, luminal microneedles are bodies that include a lumen therein.

The lumen in this class of microneedle may be used to deliver compounds (especially in connection with various reservoir means such as described in U.S. Pat. Nos. 3,964,482 or 8,257,324). Alternatively, the lumen may be used for analyte collection, in which case, an array of microneedles may be combined with analyte measurement systems to provide a minimally invasive fluid retrieval and analyte sensing system such as described in U.S. Pat. No. 6,749,792. Notable analytes of interest obtained from biological fluids include glucose and cholesterol.

Further review of microneedle technology is presented in Y. C. Kim, J. H. Park and M. R. Prausnitz, “Microneedles for Drug and Vaccine Delivery,” Advanced Drug Delivery Reviews 64 (2012). In comparison to other microneedle architectures, the hollow microneedle offers a more flexible delivery scheme as it is the only architecture that allows for advective drug delivery into the skin. The hollow microneedle is the only microneedle architecture that allows for direct use of existing formulated liquid drugs used in hypodermic injection. Additionally, drug flow through a hollow microneedle can be tailored to provide a delivery rate ranging anywhere from rapid injection (to mimic a hypodermic injection) to slow infusion similar to intravenous drug therapy.

Despite the advantages of using hollow microneedles, the complexity of fabricating the microneedles out of materials such as silicon or metals has impeded wide-scale adaptation of this technology. Even with known CNT needle manufacturing techniques (e.g., as co-invented by the inventors hereof), improvements are possible.

### SUMMARY

Systems and devices having composite CNT microneedle arrays, as well as methods of manufacturing and using the same, are described herein. These CNT microneedle arrays can provide a self-administered, painless alternative to standard hypodermic injection.

Specifically, CNT microneedle and microneedle array fabrication is contemplated in which starting-material (or “precursor”) vertically-aligned carbon nanotube pillars (VA-CNTs) are impregnated with a polymer. A thermosetting polymer resin such as polyimide may advantageously be used for such purpose. Nevertheless, thermoplastic (alternatively referred to as thermosoftening plastic) material may be used for CNT impregnation. Through the impregnation process and subsequent curing, an approach is provided that simplifies the fabrication process of microneedle arrays and/or devices to improve the prospects for scaling and commercialization.

Compared to other approaches for creating hollow microneedles using conventional materials such as silicon or metal, the subject microneedles can be fabricated rapidly as the subject approach does not rely on iterative etching techniques to define the needle shape. Instead, common techniques for growing VA-CNT are employed including catalyst patterning and chemical vapor deposition to create the needle shape.

The embodiments described herein include products produced by the processes described herein. The embodiments also include devices including products with the physical characteristics described as well as systems incorporating the same and other features as described and incorporated by reference. In addition, the present embodiments include methods of use. Such methods of use may include methods of drug delivery, of inoculation or vaccination, of analyte acquisition and analysis, etc. Suitable hardware is presented for some of such activity, whereas other hardware is incorporated by reference or would be apparent to those with ordinary skill in the art.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The figures provided herein are diagrammatic and not necessarily drawn to scale, with some components and features exaggerated and/or abstracted for clarity. Variations from the embodiments pictured are contemplated. Accordingly, depiction of aspects and elements in the figures are not intended to limit the scope of the claims, except when explicitly stated as such.

FIGS. 1A-B are Scanning Electron Microscopy (SEM) images (an overview and detail view) of an example embodiment of composite microneedles.

FIG. 1C is an SEM image of the underside of an embodiment of a microneedle demonstrating a lumen after processing.

FIGS. 2A and 2B are SEM images of example embodiments of bare and polymer-impregnated CNT elements, respectively.

FIG. 3A is a flowchart detailing an example embodiment of fabrication of composite microneedles and/or devices.

FIGS. 3B-F are diagrams depicting example stages of fabrication of composite microneedles.

FIG. 4A is a photograph of an example embodiment of a microneedle array including a polymer base acting as a secondary, connective substrate.

FIG. 4B is a photograph of the example microneedle array of FIG. 4A incorporated in an injection device.

FIG. 5A is a photograph of multiple locations of in vitro pig skin penetration and delivery of dye with an example embodiment of a microneedle array device and FIG. 5B is an enlarged view of one of the locations of FIG. 5A.

FIG. 5C is an SEM image of the example array used in producing the results of FIG. 5A.

FIGS. 6A and 6B illustrate example embodiments of medical devices with composite microneedle arrays.

### DETAILED DESCRIPTION

The present subject matter is not limited to the particular embodiments described, as those are only examples and may, of course, vary. Likewise, the terminology used herein is for the purpose of describing particular embodiments only, and is not intended to be limiting, since the scope of the present disclosure will be limited only by the appended claims.

Moreover, the teachings, techniques and other features described in U.S. Pat. No. 7,955,644 and/or US Publication Nos. 2010/0196446, 2011/0250376, 2012/0021164, 2012/0058170 and 2013/0178722 and/or each of B. Lyon, A. I. Aria, et al, APS DFD Meeting, “Carbon Nanotube Micro-Needles for Rapid Transdermal Drug Delivery,” San Diego, Calif., Nov. 18, 2012; B. Lyon, A. I. Aria, M. Gharib, “Feasibility Study of Carbon Nanotube Microneedles for Rapid Transdermal Drug Delivery,” Mater. Res. Soc. Symp. Proc. Vol. 1569, DOI: 10.1557/opl.2013.803; B. Lyon, A. I. Aria, M. Gharib, “Carbon Nanotube-Polyimide Composite Microneedles for Rapid Transdermal Drug Delivery”, Society for Biomaterials Meeting, Boston, Mass., April 2013 and B. Lyon, A. I. Aria, M. Gharib, “Feasibility Study of Carbon Nanotube Microneedles for Rapid Transdermal Drug Delivery”, MRS Spring Meeting, San Francisco, Calif., April 2013 (all of which are incorporated herein by reference in their entireties) may be employed and claimed in connection with the embodiments described herein.

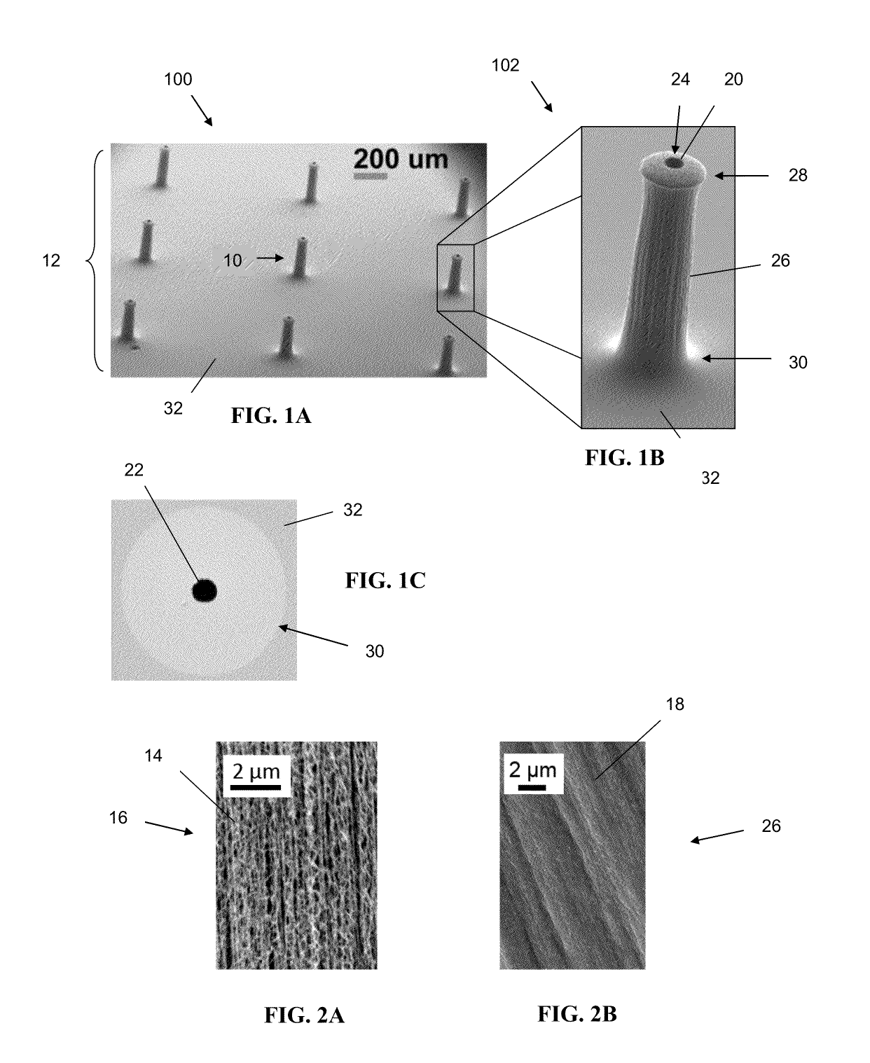

Regarding details of the subject processes and products, FIGS. 1A-C depict an example embodiment of a product with hollow microneedles fabricated using a composite of VA-CNTs and polyimide polymer. Here, SEM images provide an overall view in FIG. 1A and a detailed view in FIG. 1B of the subject composite microneedles 10. These are arranged in a regularly-spaced array 12.

Notably, VA-CNTs and other self-assembly nanomaterials allow for direct access to the nano- and micro-length scales for fabricating microneedles. Hollow bundles of VA-CNTs naturally have the high aspect ratio and micrometer scaling required for creating microneedles. As pictured, the needles are approximately 230 μm tall and 80 μm in (average) diameter with a lumen diameter of about 25 μm. As such, they exemplify about a 3:1 aspect ratio. Embodiments of the microneedles 10 may range from about 800 μm to about 150 μm in height and about 300 μm to about 10 μm in diameter and range in aspect ratio from about 10:1 to about 1:1 and be useful. They may have a lumen therein the lumen between about 10 μm and about 120 μm in diameter.

Finished microneedles may be substantially cylindrical in shape (as shown) or shaped otherwise (e.g., triangular, square, etc. in cross-section). Variability is contemplated in terms of tip “sharpness” or sharpening as in producing conical or semi-conical geometries as well. Such tapered geometry reduces the mechanical stress on the needle during skin penetration allowing for the creation of microneedles with aspect ratios of 6:1 and higher. As shown in FIG. 1B, the CNT pillar 26 tapers (decreases in width) as the height increases from pillar base 30 towards pillar tip 28. The taper reverses upon reaching pillar tip 28 which flares outward compared with the lower pillar structure. The uppermost termination of pillar 26 is generally rounded, but can also be flat.

As shown in FIGS. 1A-C, a top port 20 may be the same size (or different in size) than a bottom port 22 in the needle with lumen 24 running there-between through a pillar or column section 26. The lumen may be substantially uniform in diameter or tapered in diameter. The lumen will be (at least) substantially uniform if there is no shrinkage in the size of the microneedle during processing caused by polymer resin evaporation. If there is shrinkage, the lumen will be tapered as the decrease in outer diameter causes the lumen to shrink as well.

In any case, a lumen provided in a precursor VA-CNT structure can be maintained intact in connection with the polymer-composite processing as described below between a pillar or column tip 28 and a pillar or column base 30 as well as through a substrate base 32 to which base 30 is attached or otherwise incorporated therein.

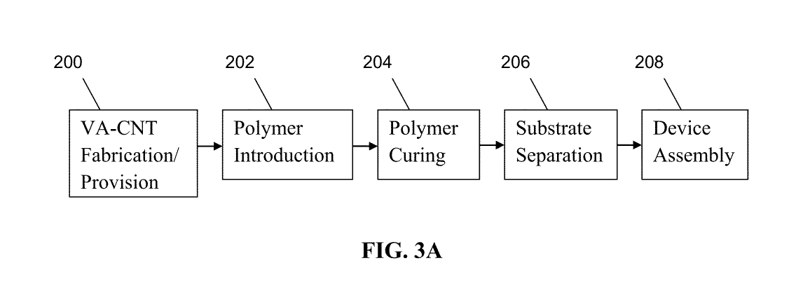

Regarding such processing, FIGS. 2A and 2B are illustrative. In one embodiment, VA-CNT/polyimide composite microneedles are fabricated by wicking polyimide resin passively through and into fibers (or nano-fibers) 14 of a CNT bundle 16 then polymerizing the polyimide resin 18 thermally to create one or more functioning hollow microneedle pillars or columns 26. Polyimide is advantageously chosen for composite processing due to its ability to conformally coat the VA-CNTs during the wicking process and because of its inherent stiffness (approximate Young's modulus of 3 GPa). It also provides advantages by providing a flexible base material as further described below.

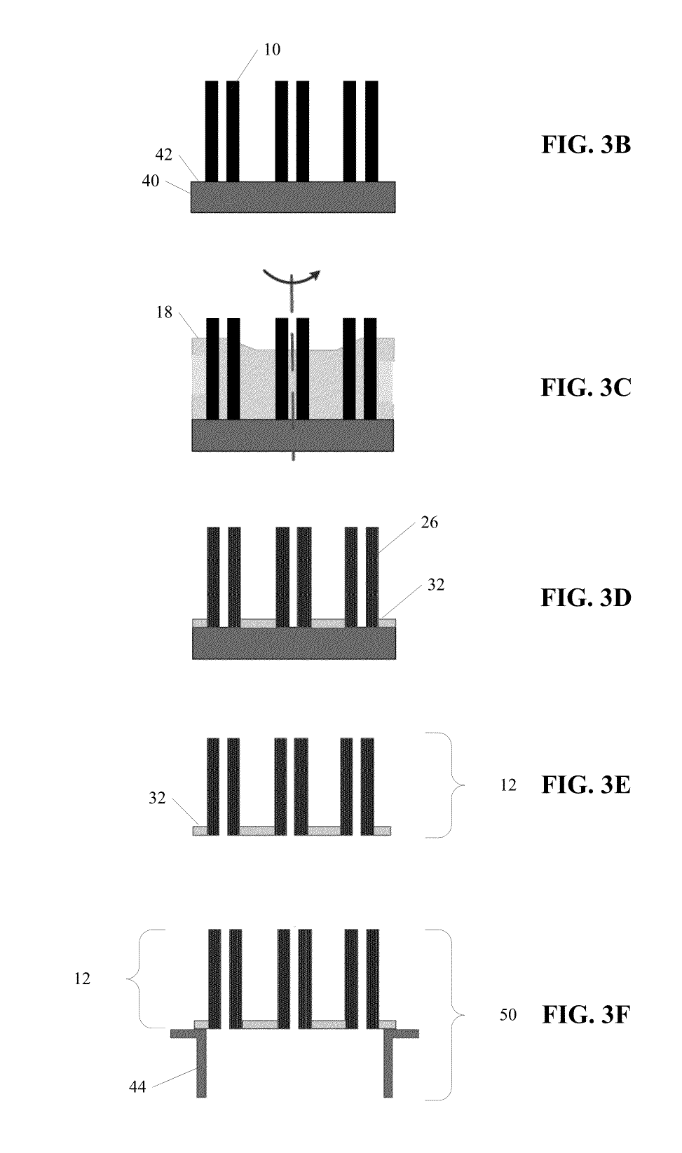

FIG. 3A depicts an example embodiment of a fabrication process that may begin at 200 with VA-CNTs fabrication or provision. The CNTs may be fabricated as described in the above-incorporated references and/or modified after fabrication (but prior to polymer introduction, etc. as further described below) such as described in the above-referenced US Pub. No. 2013/0178722, “Sharp tip CNT Microneedle Devices and Their Fabrication.”

At 202 a polymer is introduced into the CNT precursor structure. The polymer can be incorporated via drop-casting, followed by spin-coating. In this (combined) step of polymer introduction, drop-casting can be performed by pouring liquid polymer over the sample until the sample is completely immersed. During drop-casting, polyimide resin begins to wick into the CNTs and clogs each lumen. Spin coating can be initiated after drop casting. During spin coating, three results occur simultaneously: 1) wicking of polyimide resin into CNT structures; 2) clearing the lumens by resin moving from the lumens into the CNT structures; and 3) forming a common polymer base to unite the array into a single structure to allow for later separation from the substrate upon which it is produced. The polymer can then be cured at 204, solidifying the CNT pillars or columns and common base.

Optionally, (e.g., in connection with polyimide resin) the curing is a thermal curing cycle or process. Once cured, the resulting composite microneedle array is separated from a substrate on which it was formed at 206. Such activity may be followed by incorporating the array into an overall medical device at 208 (e.g., by connecting, affixing or otherwise associating the array's base with a backing, housing or medical device base). Various medical device examples are further discussed below.

As further illustrated in FIG. 3B, VA-CNT precursor bundles 16 may be fabricated on a silicon wafer substrate 40. Substrate 40 may be coated with 1 nm iron catalyst 42 that is patterned into hollow rings (e.g., 150 μm outer dia., 25 μm inner diameter) through photolithography and electron beam evaporation. During chemical vapor deposition, ethylene and hydrogen gas interact with sintered catalyst nanoparticles to form VA-CNTs with approximate diameter of 25 nm with VA-CNTs can be grown to heights ranging from 100 μm to 800 μm.

In FIG. 3C, polyimide monomer 18 is drop-casted and then spin coated onto the CNTs to simultaneously create a uniform composite of CNT and polymer, clear the lumen of polymer resin and simultaneously creating a flexible polymer substrate base for the array. Microneedles 10 ultimately adapt the shape of the VA-CNT bundle as the polyimide resin 18 conformally coats the VA-CNT bundle and fills the interspacing between individual VA-CNTs.

FIG. 3D depicts an example curing step. In some instances depending on the procedure to cure the polyimide monomer at 18, solvent evaporating from the uncured polyimide causes the VA-CNT structure to contract allowing microneedles 10 to be reduced in diameter while simultaneously increasing the packing density of microneedles 10 in an array. For lightly viscous polyimide monomer solutions, curing can be done thermally without clogging the central cavity. Moreover, UV curing procedures (e.g., in connection with using epoxy resin to impregnate the CNTs and form their common base) may be employed.

In one example, Poly(3,3″,4,4″-benzophenonetetracarboxylic dianhydride-co-4,4″-oxydianiline/1,3-phenylenediamine), i.e., BOPDA polyimide, was applied by spin coating. After the spin coating, the composite was cured at 100° C. for 1 hour followed by 30 minute curing at 150° C. and 200° C. Finally, the composite was left to cure at 250° C. for several hours to allow for complete imidization of the polymer.

In the case of BOPDA polyimide, contraction of the VA-CNT structure during polymer curing allows for creation of 80 μm outer diameter microneedles from an original VA-CNT precursor structure with an outer diameter of 150 μm.

Other suitable liquid plastics with which to impregnate the VA-CNT bundles include HD-4110 polyimide, KAPTON, THORLON and SU-8 epoxy. Whether polyimide polymer or another, the material for coating may have a viscosity between about 8 cSt and about 8,000 cSt for thermal curing. It is also contemplated that thermoplastic particles such as high-strength PEEK can be dissolved in a thermoset polymer such as BOPDA or Kapton to reinforce the CNT-polymer composite. For using a thermoplastic directly during spin coating, that can be achieved by placing the CNT sample on a heated surface during spin coating.

Whether a thermosetting resin or thermosoftening plastic is used, a characteristic wicking velocity defined as the ratio of surface tension to viscosity can be used to screen potentially useful polymers and/or thermoplastic processing temperatures. A material for coating may have a characteristic wicking velocity between 0.2 cm/s to 500 cm/s. A selective curing or UV approach can be used for polymers of greater viscosity. In any case, stiffness of the cured (or cooled/solidified in the case of thermoplastic material) polymer should be greater than 1 GPa to ensure the resulting composite microneedles are capable of penetrating a subject's skin.

In FIG. 3E, the polymer substrate base 32 binding and carrying composite microneedles 10 is removed from the silicon substrate 40 resulting in a free array 12. In this example, poor mechanical adhesion between polyimide and silicon allow the resulting product to be easily removed from the silicon mechanically using tweezers and/or a razor blade. Finally, in FIG. 3F, array 12 is attached to a delivery device backing, base or shell element 44 to create a medical device or medical device sub-assembly 50.

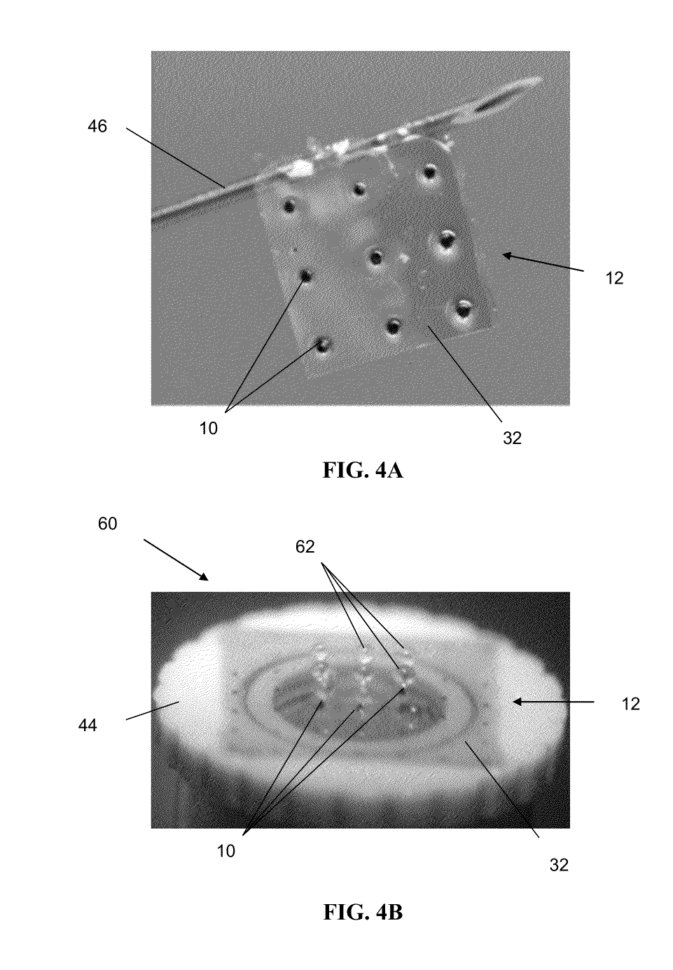

FIG. 4A illustrates and exemplary microneedle array 12 produced as described above (in this case hanging on 30 Gauge hypodermic needle 46). Here, an example manner in which the polymer base 32 maintains association and organization of the finished CNT pillars can be seen. The base pictured is about 50 μm thick. Polymer bases as produced and employed herein may range from about 10 μm to about 100 μm thick. They facilitate handling as well as medical device fabrication as shown, described and as may be further appreciated by those with skill in the art.

In FIG. 4B, array 12 is shown incorporated in an injection device 60. Specifically, array 12 is set upon a backing element 44. With the backing element attached to a syringe (not shown) deionized water was pushed through the nine-needle array shown at 20 uL/min per needle. Static water droplets 62 are shown perched atop each needle 10 in the array as produced in connection with such testing.

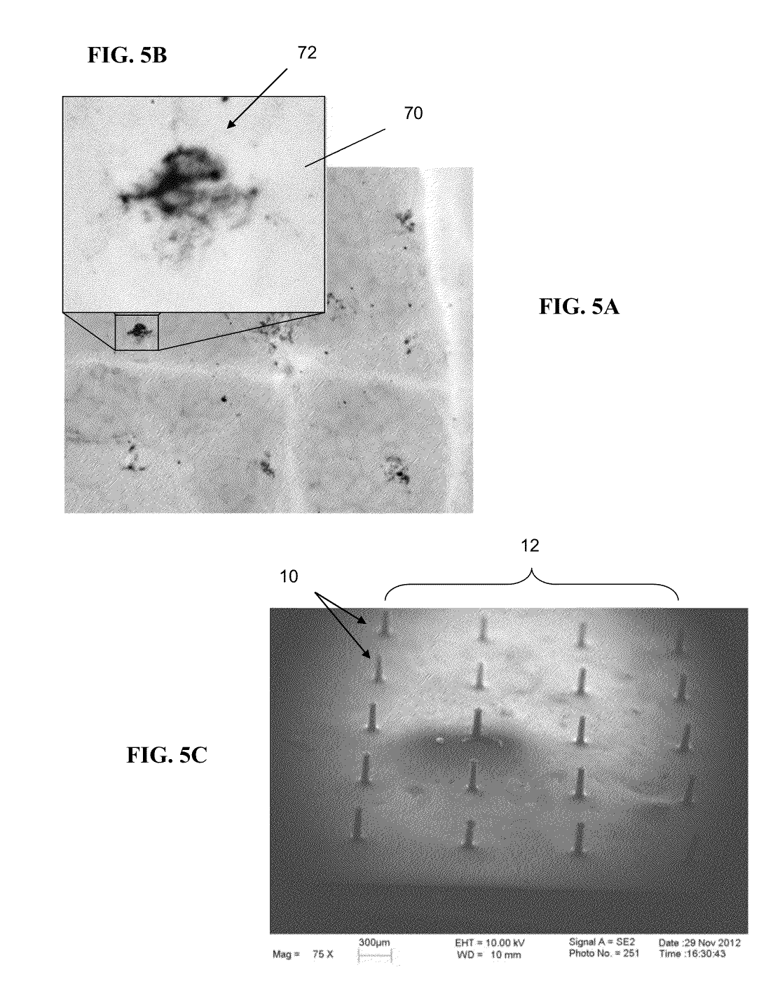

FIGS. 5A-B are pictures depicting the results of in vitro testing via porcine skin 70 penetration and delivery of methylene blue dye 72. Such results were produced using 100 μm diameter polyimide-impregnated VA-CNT needles. Skin penetration was optimally achieved without damaging the microneedles employing a needle length/height in the range of 200 μm-300 μm. FIG. 5C is an SEM image of such an array 12 as used for the skin penetration testing wherein the VA-CNT Polyimide Composite microneedles 10 had a mean needle height of 230 μm. As seen, the (used) needles in the array show no signs of breaking, buckling or other structural damage.

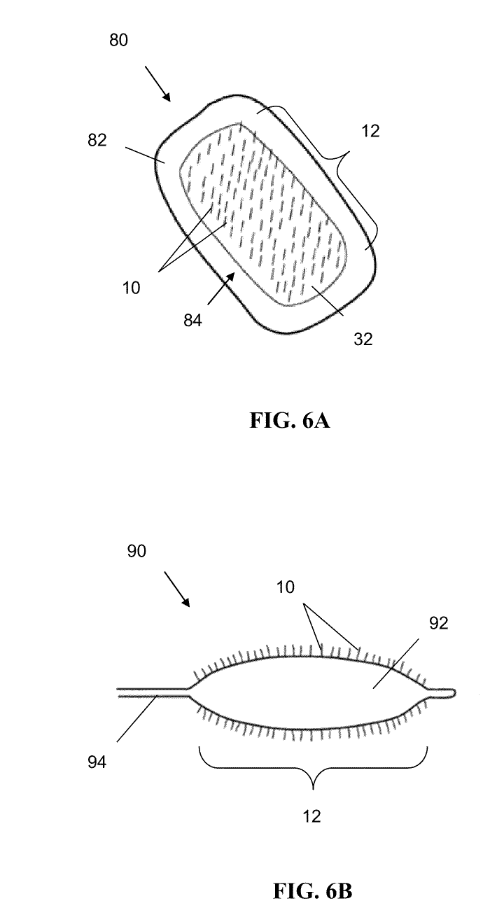

Employing such technology, a variety of medical devices can be produced. FIG. 6A is an example embodiment of an epidermal patch 80 to be worn on the skin of a subject. Patch 80 has aligned microneedles 10 protruding from a base 32 in a microneedle array 12 to be adhered to the skin. A portion of the patch, for example the perimeter of a flexible backing or cover 82 to which base 32 may be attached is optionally coated with adhesive to facilitate releasably securing the device to the skin. When patch 80 is applied to the skin, microneedles 10 pass through the stratum corneum and enter the viable epidermis to release any drug or agent that may be wicked or driven through the needles from a reservoir 84 (e.g., as in a pocket or a porous medium) held by the patch behind the substrate. Another suitable epidermal patch that may incorporate the subject microneedle technology is described in U.S. Provisional Patent Application Ser. No. 61/876,072 filed Sep. 10, 2013, which is incorporated by reference herein its entirety for all purposes.

FIG. 6B illustrates an example embodiment of angioplasty device 90 with a balloon 92 at the end of a shaft 94. More specifically, balloon 92 is shown inflated and bearing an example microneedle array 12 for delivering an anti-hyperproliferation drug (e.g. paclitaxel) through microneedles 10 following the inflation of the balloon to open a section of a blood vessel. Alternatively, such a balloon (or another structure) could employ the subject composite microneedles 10 for delivering any of a variety of substances to various parts of the body, including, but not limited to, the skin, uterus, bronchial tubes, and various portions of the gastrointestinal tract including the colon. In any case, the exemplary thin flexible polyimide base 32 of array 12 allows for easily incorporating microneedles into curved, plastic surfaces such as by affixation of the array base to the balloon surface to provide a backing thereto.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Express recitation of every possible combination and substitution is overly burdensome, especially given that the permissibility of each and every such combination and substitution will be readily recognized by those of ordinary skill in the art upon reading this description.

As used herein and in the appended claims, the singular forms “a”, “an”, and “the” include plural referents unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A method of polymer carbon nanotube (CNT) needle array fabrication comprising:
providing a plurality of CNT bundles in an array upon a substrate;
coating the substrate and impregnating the bundles with a liquid polymer;
curing the polymer to produce polymer composite CNT pillars embedded in a polymer base; and
removing the base from the substrate.

2. The method of claim 1, wherein the liquid polymer comprises thermoset resin.

3. The method of claim 2, wherein the liquid polymer is polyimide.

4. The method of claim 2, wherein the liquid polymer is Poly(3,3″,4,4″-benzophenonetetracarboxylic dianhydride-co-4,4″-oxydianiline/1,3-phenylenediamine)polyimide.

5. The method of claim 1, wherein the liquid polymer comprises a heated thermosoftening polymer.

6. The method of claim 1, wherein the CNT pillars are between about 10 μm and about 300 μm in diameter.

7. The method of claim 6, wherein the CNT bundles are larger in diameter than the CNT pillars after curing.

8. The method of claim 1, wherein the CNT pillars include a lumen therein.

9. The method of claim 8, wherein the lumen is between about 10 μm and about 120 μm in diameter.

10. The method of claim 8, wherein the CNT pillars are between about 150 and about 800 μm in height.

11. The method of claim 1, wherein the base is between about 10 μm and about 100 μm thick.

12. The method of claim 1, further comprising connecting the base to a backing of a medical device.

13. A microneedle array device, comprising:
a plurality of vertically-aligned carbon nanotube pillars comprising carbon nanotube bundles and a polymer,
wherein the polymer forms a base connecting the pillars, and
wherein the base is connected to a medical device backing element and is free from any substrate on which the base was produced.

14. The device of claim 13, wherein the pillars include a lumen therein.

15. The device of claim 14, wherein the lumen is between about 10 μm and about 120 μm in diameter.

16. The device of claim 13, wherein the pillars are between about 10 μm and about 300 μm in diameter.

17. The device of claim 13, wherein the pillars are between about 150 μm and about 800 μm in height.

18. The device of claim 13, wherein the base is between about 10 μm and about 100 μm thick.

19. The device of claim 13, wherein the backing element is part of a dermal patch.

20. The device of claim 19, further comprises a drug-filled reservoir.

21. The device of claim 13, wherein the backing element is part of a balloon catheter.

22. The device of claim 13, wherein the polymer is a thermoset polymer.

23. The device of claim 22, wherein the polymer is polyimide.

23. The device of claim 22, wherein the polymer is Poly(3,3″,4,4″-benzophenonetetracarboxylic dianhydride-co-4,4″-oxydianiline/1,3-phenylenediamine)polyimide.
